(19) United States
(12) Patent Application Publication
(10) Pub. No.: US 2004/0059184 A1
(43) Pub. Date: Mar. 25, 2004

(54) Alternative therapy method

(76) Inventor: Montsho S. Badarinwa, Laurel, NJ (US)

Correspondence Address: Richard C. Litman, LITMAN LAW OFFICES, LTD., P.O. Box 15035, Arlington, VA, 22215, US

(21) Appl. No.: 10/235,485

(22) Filed: Sep. 6, 2002

(51) Int. Cl. A61M 21/00

(52) U.S. Cl. 600/26

(57) ABSTRACT

An alternative therapy method for pain management and healing, performed by a therapist on a patient. Steps of the overall method include preparing the therapist, the patient and their environment for a therapeutic session, assessing the patient's spiritual energy field, having the therapist explain to the patient what procedures will be done during the therapeutic session, obtaining verbal permission from the patient to perform the procedures during the therapeutic session, facilitating the patient's flow of spiritual energy, repatterning the patient's areas of spiritual imbalance and impeded energy flow, encouraging the patient's feedback and documenting the results of the therapeutic session and the patient's feedback.

### BACKGROUND OF THE INVENTION

1. Field of the Invention

The present invention relates to an alternative therapy method of pain management and treatment. The method involves the combination of a number scientific disciplines such as yoga, massage, metaphysics, prayer, traditional Western medicine and others into the steps of a single therapeutic method.

2. Description of the Related Art

Pain management and healing can involve not only the science of modern medicine, but can also involve lesser known sciences and methodologies. Spiritual counseling, yoga, massage and various other treatments can also be used in a pain management and healing program. A broader and a more “holistic” approach to medicine can be taken and blended with modern medicine. Some of these methodologies are reflected in the related art.

U.S. Pat. No. 5,147,294 issued to Smith et al., outlines the use of a therapeutic methodology for the treatment of chronic pain, from its earliest stages to the most severe and intractable stage in a living subject. The therapeutic methodology is able to provide substantial pain relief over a long period of time to patients afflicted with chronic pain and an improvement of all symptoms coincidental with chronic pain. The therapeutic procedure must be performed by a properly trained medical practitioner or clinician within the confines of a hospital setting.

U.S. Pat. Nos. 5,676,633 and 5,681,259 issued to August, outlines the use of an apparatus and method that promotes patient relaxation, reduces patient stress and expedites patient recovery in a health care facility or home-based convalescent environment. The apparatus includes a substantially glare-free visual display device with one or more open, natural, serene and savanna-like landscape pictures. These pictures include large high resolution photo-realistic images, which are displayed a substantially glare-free display device.

U.S. Pat. No. 5,930,851 issued to Brunelle, outlines the use of a hydro-thermo massaging tub and method of massage treatment wherein the treatment is performed by warm air jets distributed adjacent the bottom wall of the massaging tub all about its circumference. Warm air jets are released in the water contained within the tub to impart turbulence in the body of water to create acupuncture massaging flows of warm air jets and water flows from opposed sidewalls and end walls of the tub.

U.S. Pat. No. 6,000,828 issued to Leet, outlines the use of a computer implemented method and system for improving drug treatment of patients in local communities by providing drug treatment protocols for particular disease states, such as Diagnosis Related Group (DRG) classifications. The protocol contains ranked recommendations for drug treatments of the diseased state, and the computer system collects information about the risks and benefit of the drug treatments. The information collected about the treatments is used to modify the rankings of the drug treatments in the protocol.

U.S. Pat. No. 6,208,902 issued to Boveja, outlines the use of an apparatus and method for adjunct therapy of painful syndromes, which include an implantable lead-receiver, an external stimulator having controlling circuitry and a power source, and a coil to inductively couple the stimulator to the lead-receiver. The external stimulator emits electrical pulses to stimulate a cranial nerve such as the left vagus nerve, according to a predetermined program. The predetermined program include both short-term and long-term stimulation.

U.S. Pat. No. 6,241,693 issued to Lambden, outlines the use of an apparatus and a method for applying pressure to the body of a person. The apparatus is made up of an arcuate shaft, a handle that extends from the arcuate shaft in a plane intersecting the plane of the arcuate shaft and a pressure applicator movably attached to the arcuate shaft for applying pressure to the body. The apparatus can be used for massaging or for applying acupressure.

U.S. Pat. No. 6,306,077 issued to Prabhu et al., outlines the use of a method of managing a psychological state of an individual using images which include determining an individual's direction or preference for a state management session. This is based on whether a current set of images selected through a personal image profile session will achieve the desired management effect. If the current set are deemed satisfactory, they are presented to the individual to achieve management of the individual's state.

U.S. Pat. No. 6,378,138 issued to Ridgley et al., outline the use of a stocking or hosiery which incorporates various types of healing devices, such as crystals, for healing various bodily ailments. The naturally occurring elements may be incorporated into various locations of pantyhose or stockings, including the waistband, the area immediately below the waist, the knees, and ankle bands and the outline areas of the pantyhose.

Although each of the patents outline useful and novel pain management and healing treatment methods, what would really be of use to people who are in need of pain management and healing services are methods that utilize a broader “holistic” scope of therapies and protocols in addition to modern Western medicine. These therapies can include various massage techniques, yoga, music therapy and aromatherapy.

None of the above inventions and patents, taken either singularly or in combination, is seen to describe the instant invention as claimed. Thus a alternative therapy method solving the aforementioned problems is desired.

### SUMMARY OF THE INVENTION

The invention is an alternative therapy method for pain management and healing, performed by a therapist on a patient. Steps of the overall method include preparing the therapist, the patient and their environment for a therapeutic session, assessing the patient's spiritual energy field, having the therapist explain to the patient what procedures will be done during the therapeutic session, obtaining verbal permission from the patient to perform the procedures during the therapeutic session, facilitating the patient's flow of spiritual energy, repatterning the patient's areas of spiritual imbalance and impeded energy flow, encouraging the patient's feedback and documenting the results of the therapeutic session and the patient's feedback.

Accordingly, it is a principal object of the invention to provide a holistic pain management and healing therapy.

It is another object of the invention to provide a pain management and healing therapy that utilizes various massaging techniques.

It is a further object of the invention to provide a pain management and healing therapy that utilizes a wide variety of alternative treatments and protocols.

Still another object of the invention is to provide a pain management and healing therapy that uses metaphysical procedures.

It is an object of the invention to provide improved elements and arrangements thereof for the purposes described which is inexpensive, dependable and fully effective in accomplishing its intended purposes.

These and other objects of the present invention will become readily apparent upon further review of the following specification and drawings.

### BRIEF DESCRIPTION OF THE DRAWINGS

FIG. 1 is an outline of the steps of an alternative therapy method of pain management and treatment, according to the present invention.

FIGS. 2A, 2B, 2C and 2D make-up a protocol table for specific diagnoses.

FIG. 3 is a diagram of a chakra table indicating the locations of specific chakras on the human body.

FIGS. 4A and 4B make-up a health information form for patients or physicians to complete before being subjected to an alternative therapy method of pain management and treatment.

FIG. 5A is a listing of essential oils and incense used for specific treatments.

FIG. 5B is a list of essential oils referred to in ancient scripture.

FIG. 6 is a listing of prayers used for addressing specific problem situations.

FIG. 7 is a listing of healing stones that are used to treat various conditions at specific chakras and other high energy areas.

FIGS. 8A and 8B make-up a chart of acupuncture meridians on the human body.

FIGS. 9A and 9B make-up reflexology charts for hands and feet.

Similar reference characters denote corresponding features consistently throughout the attached drawings.

### DETAILED DESCRIPTION OF THE PREFERRED EMBODIMENT

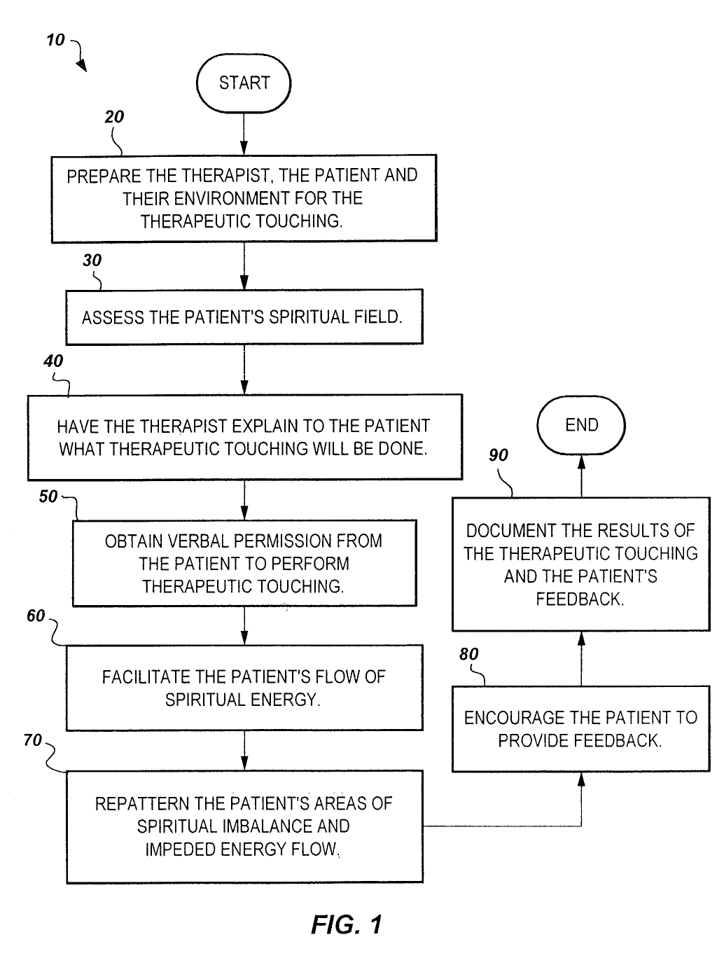

The present invention is an alternative therapy method for pain management and healing 10, performed by a therapist on a patient. The alternative therapy method for pain management and healing 10 comprises the steps of preparing the therapist, the patient and their environment for the therapeutic session 20; assessing the patient's spiritual energy field 30; having the therapist explain to the patient what procedures will be done during the therapeutic session 40 and obtaining verbal permission from the patient to perform the procedures during the therapeutic session 50; facilitating the patient's flow of spiritual energy 60; repatterning the patient's areas of spiritual imbalance and impeded energy flow 70; encouraging the patient's feedback 80; and documenting the results of the therapeutic session and the patient's feedback 90. These steps are outlined in FIG. 1.

An alternative therapist is a trained professional, such as an MD/RN/LPN/DO. This person is also “living their practice” and “walking their talk” and is living in peaceful harmony with all living creatures. These people are of all denominations and are ambassadors of truth and justice and have dedicated their lives to serving others. These people have gone “the extra mile” to spiritually develop and heal themselves and are spiritually mature and spiritually balanced before they heal others. They have spiritual commitments that can be measured easily, as evidenced by changing their name (for example), thus shedding their old former selves and lifestyles and becoming new. They have had some form of in depth counseling themselves, which must be documented.

They are or have been instructed on a one-on-one basis and can only accept a maximum of three students per year. They do not direct or dictate others lives and do not conform to only one religious practice and are universal spiritualists. They are well-versed in their own personal religious practices, as well as others, such as Buddhism or the like, but don not impose their belief systems on other in any way.

Before the therapeutic session begins, the therapist must first prepare himself or herself, as well as the patient and their environment for the therapeutic session 20. This is the first step of the overall method. Approximately 1 hour before the session, the therapist should clear his or her mind and take at least 15-20 minutes alone to meditate and pray. Once the therapist has completed the meditation and prayer, the therapist should perform a visualization technique to further clear his or her thoughts and clear any negative spiritual energy in the patient's and therapist's environment during the therapeutic session. The therapist should sit down and close his or her eyes and begin to visualize a box outside of the room or environment of the therapeutic session. The therapist should then imagine that all of his or her problems and troubles would be put in the box. Once put in the box, the therapist should then lock the box and put the key away and continue to think that the problems in the box will have to wait until after the therapeutic session.

The therapist should then begin to prepare the environment for the therapeutic session. Using incense to clear negative spiritual energy, the therapist should then ignite one of the specific incense of either Sandalwood, Frankincense or Myrrh in each corner of the environment (North, South, East and West). As the therapist places the incense, the therapist should say out loud that the environment is a safe place and that no negativity is invited here and that only the Sacred Peace of the Lord may enter here.

The therapist should then stand in the center of the environment. The therapist should then take his or her right hand, with the palm facing inward, and make a sweeping motion in the environment to sweep all of the negative energy into the therapist's hand. The therapist should say out loud that the environment is cleared of all negative energy and that the negative energy in the therapist's hand will be transmuted into positive energy into the universe. The therapist should visualize the energy as a pink ball that will float up to heaven and leave the environment. This sequence of events should be done before and after every therapeutic session.

The therapist should then prepare a manual therapy table (not shown) to perform some of the steps of the overall method. A clearing solution is sprayed to clear negative spiritual energy on the manual therapy table. The clearing solution is 4 oz. of alcohol and 6-12 drops of Lavender essential oil, which is sprayed on the table before and after every therapeutic session. A solution of ¼ cup of sea salt and 1 gal. of water should also be prepared in a large open top pot that is then placed in the corner of the environment. This is done to collect all of the negative energy that the patient is holding onto and releasing during the therapeutic session. After each therapeutic session, this water should be discarded in the toilet and the large open top pot should then never be used to eat from again.

The therapist should perform another visualization exercise before addressing the patient. This visualization exercise is to spiritually connect the therapist to the spiritual energy from the earth. The therapist should first place his or her hands in a praying position in front of his or her heart Chakra. The therapist should imagine that his or her legs are like the trunk of a tree and that his or her feet are like the roots. The tree is thought to be huge and that there is an unbreakable bond with the core of the earth. The therapist must imagine spiritual energy coming from the earth's core and that it is moving up through the body of the therapist. The sun is then imagined to radiate energy onto the top of the therapist's head and moves down through the therapist and connects with the imagined energy coming from the earth. This is to spiritually ground the therapist before beginning the therapeutic session.

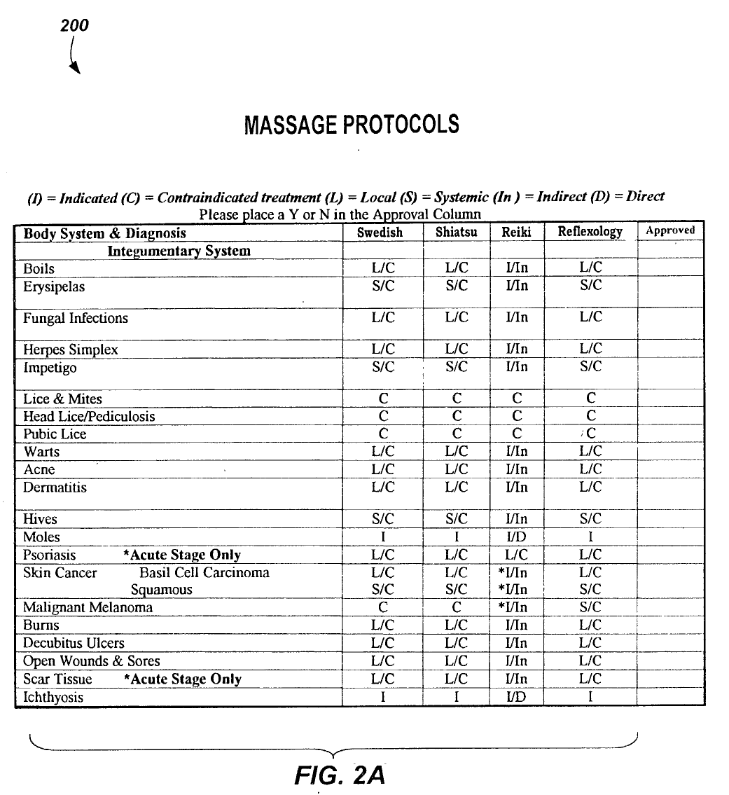

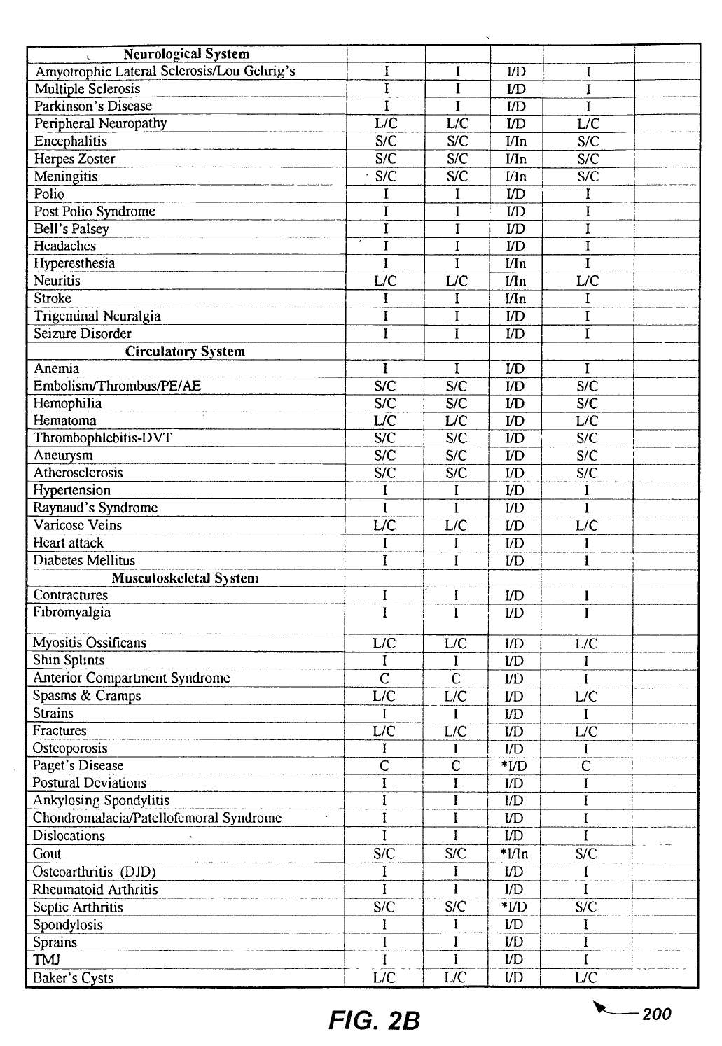

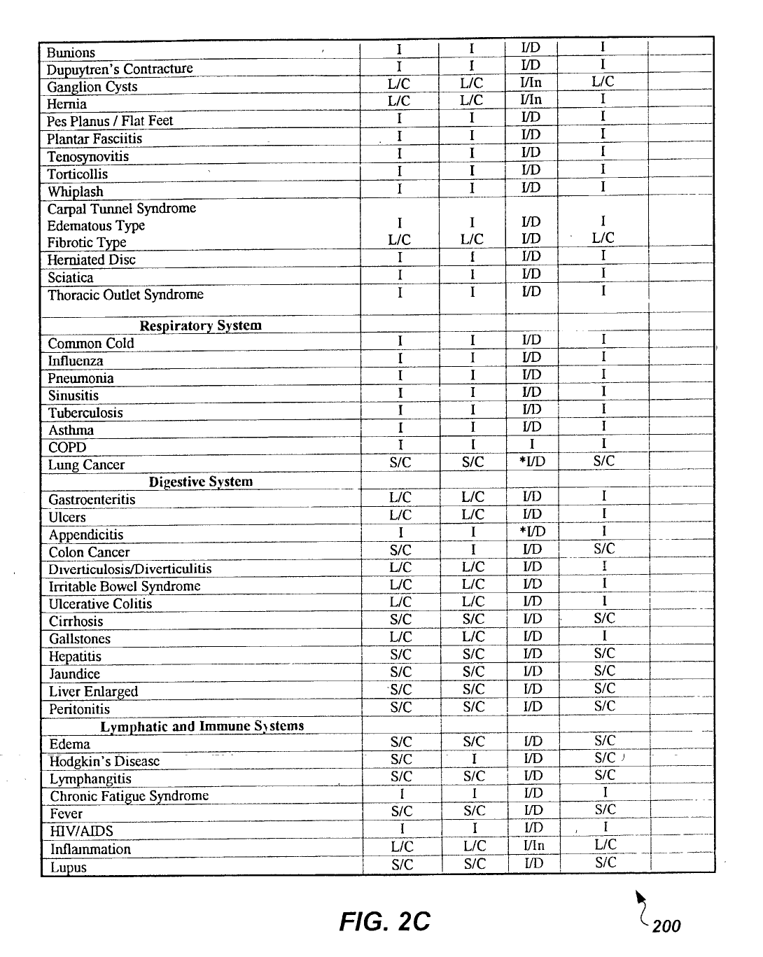

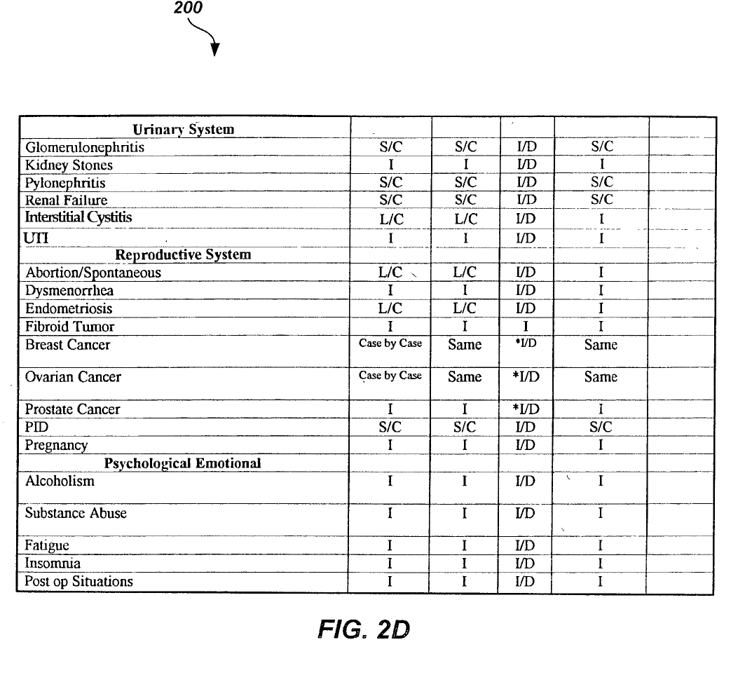

The second step of the overall method is assessing the patient's spiritual energy field 30. This is done before the beginning of the therapeutic session. The patient will first fill out the protocol table listed in FIGS. 2A, 2B, 2C and 2D and will note to the therapist any areas where they complain of pain or tension. The protocol table 200 breaks the body systems and diagnosis down into the integumentary system, neurological system, circulatory system, musculoskeletal system, respiratory system, digestive system, lymphatic and immune systems, urinary system, reproductive system and psychological system. Four different types of massage techniques are indicated for components of each of the named systems. A legend is developed where the particular type of massage treatment is indicated or contraindicated. Specific information such as local or systemic treatment is also indicated for each massage type as well as notation for indirect and direct treatments. The 4 types of massage techniques used are Swedish, Shiatsu, Reiki and reflexology.

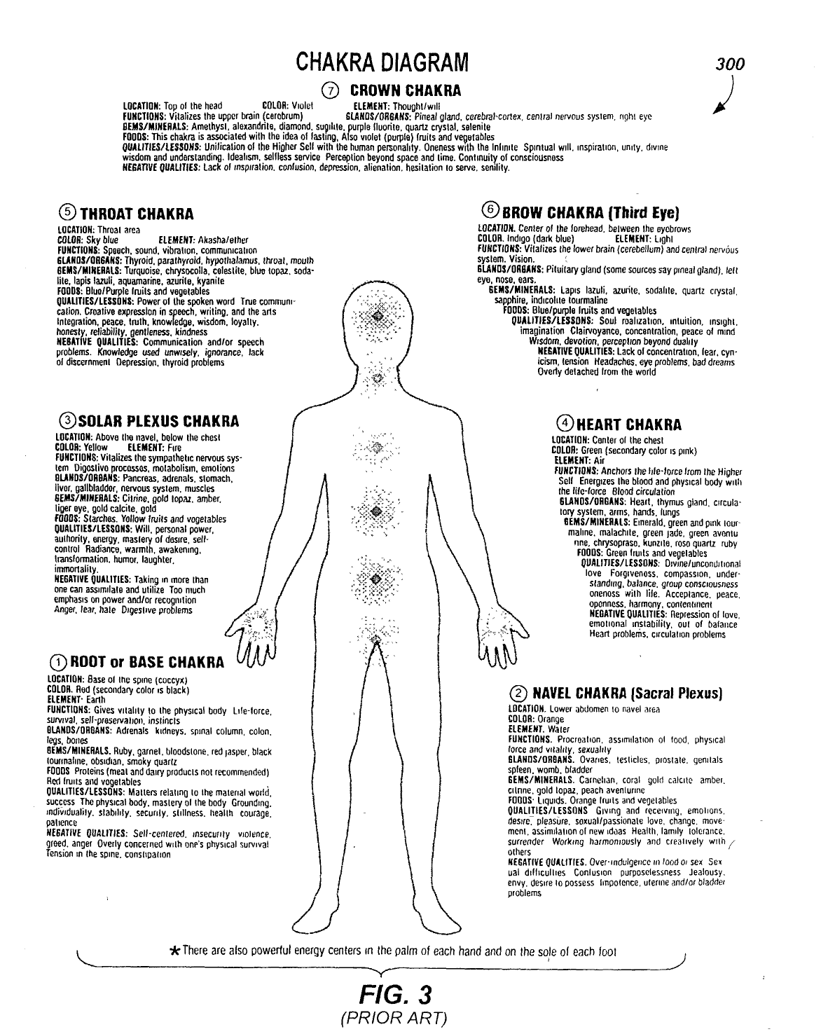

Using the Chakra table 300 on FIG. 3, the therapist will note the Chakra, organ, mental and emotional issues, physical dysfunction and energy center it corresponds to as well as the area that must be concentrated on for treatment. There are 7 “Chakras”, as indicated on the Chakra table 300. These include the root or base chakra, the navel chakra, the solar plexus chakra, the heart chakra, the throat chakra, the brow chakra and the crown chakra. The patient should also tell the therapist how they are feeling and the patient's posture and body language should also be noted. The therapist should rely on his or her gut reaction regarding the patient's body language and demeanor.

The third and fourth step of the alternative therapy method for pain management and healing 10 is having the therapist explain to the patient what procedures will be done during the therapeutic session 40 and obtaining verbal permission from the patient to perform the procedures during the therapeutic session 50. The therapist should ask the patient exactly what they would like to accomplish from the therapeutic session. The therapist should specifically ask the patient if they want their spiritual energy balanced and have any spiritual energy blockages removed. The therapist should also stress that the therapeutic session is not a cure-all. The therapist should also assure the patient that they may be faced with some feelings during the therapeutic session that will not feel good, but are necessary as part of their healing.

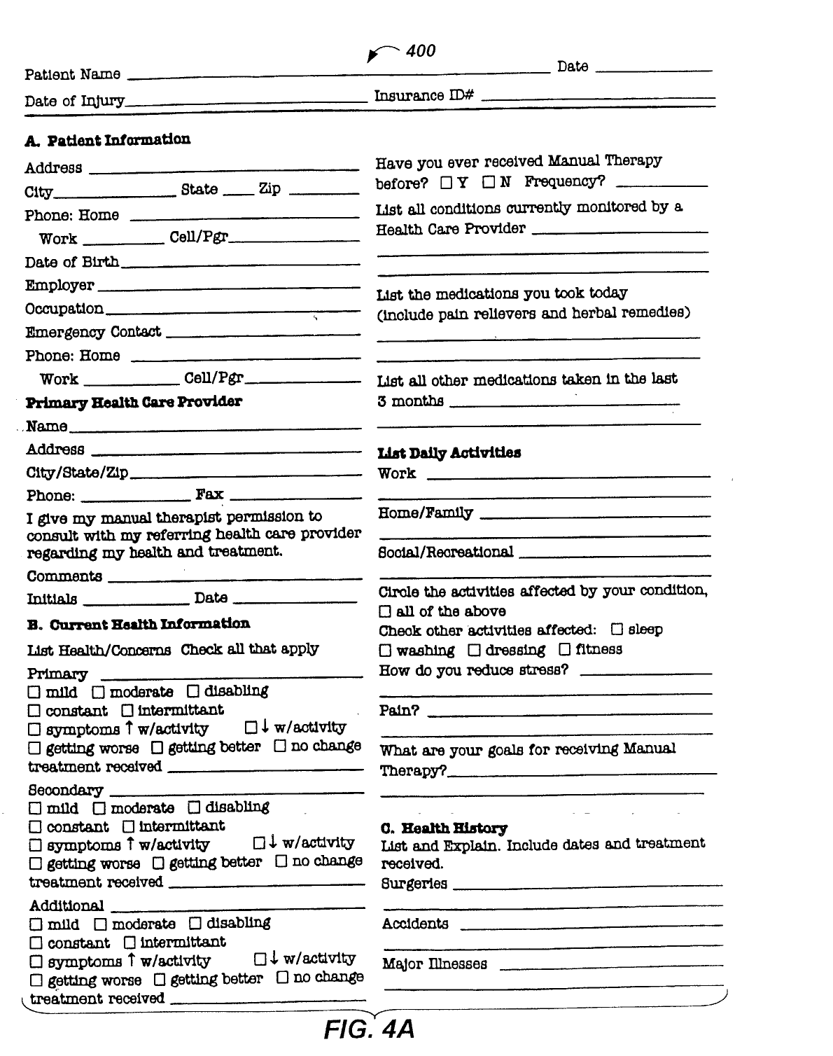

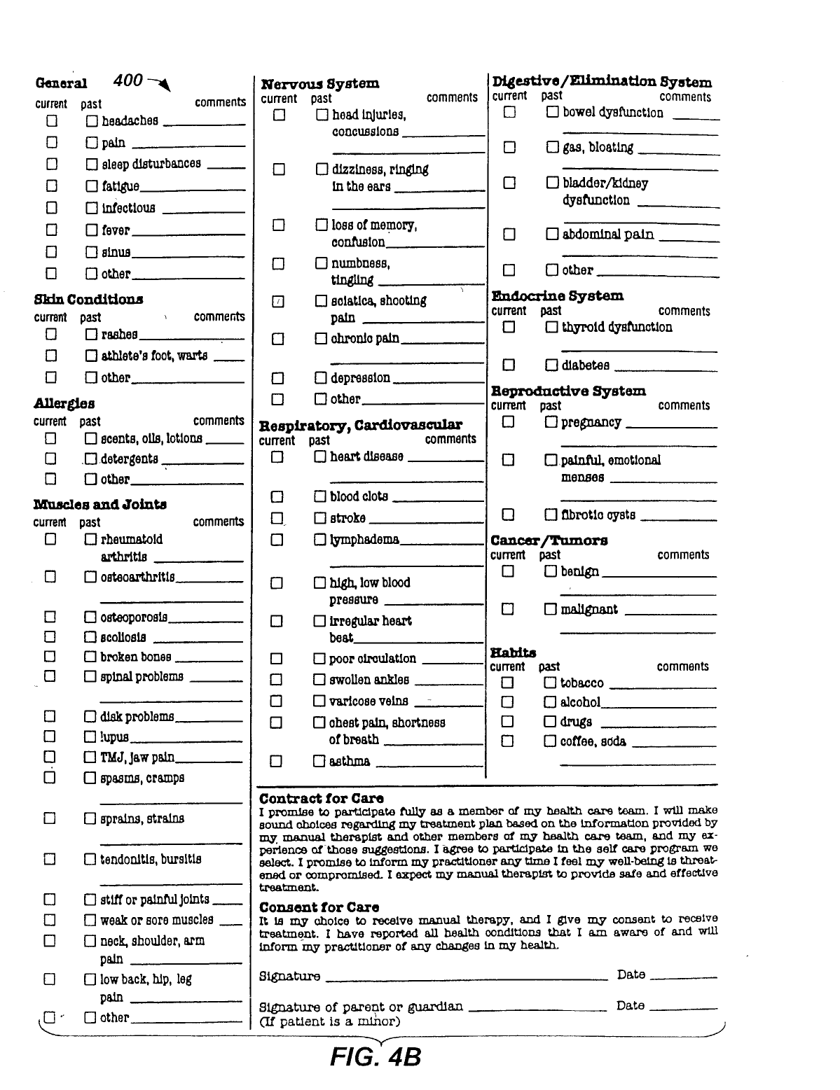

The therapist will then perform the fifth and-sixth steps of the overall method which facilitate the patient's flow of spiritual energy 60 and repatterning the patient's areas of spiritual imbalance and impeded energy flow 70. The therapist should first obtain and analyze the patient's previous medical history, using a form similar to the medical history questionnaire 400 outlined in FIGS. 4A and 4B. This medical history questionnaire 400 is filled out by the patient or a physician and includes current health information, a list of daily activities, and specific questions regarding the patient's nervous system, respiratory and cardiovascular systems, digestive system, endocrine system, reproductive system and past medical conditions,.

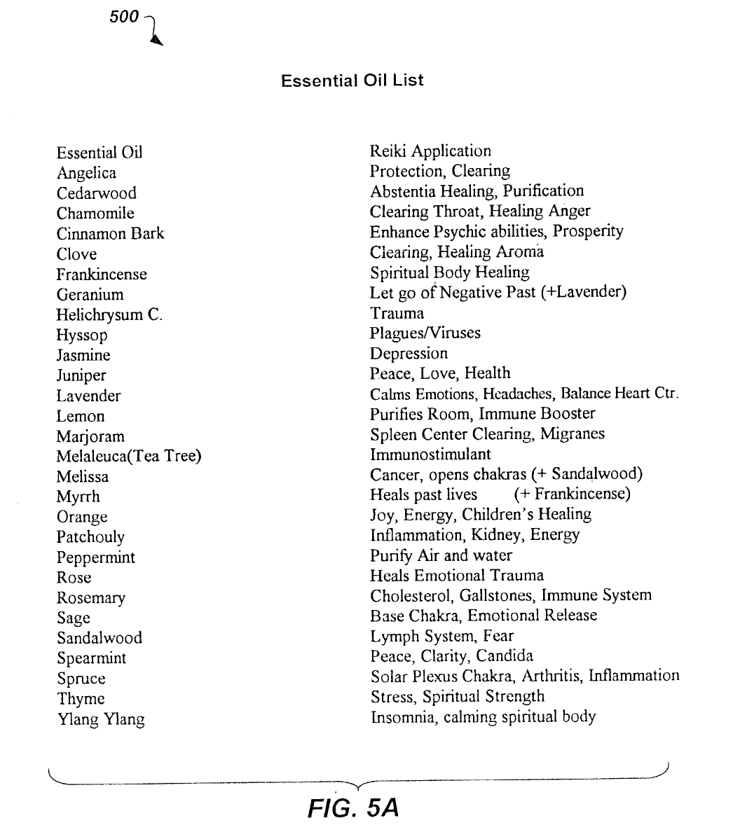

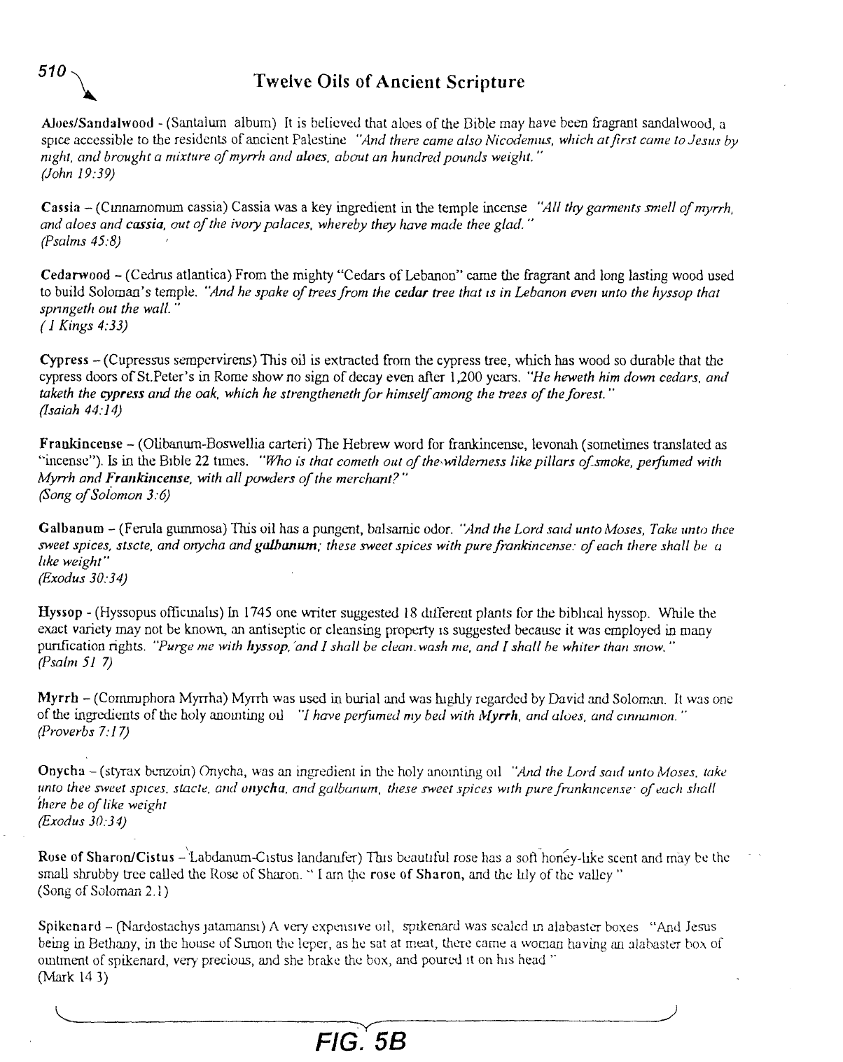

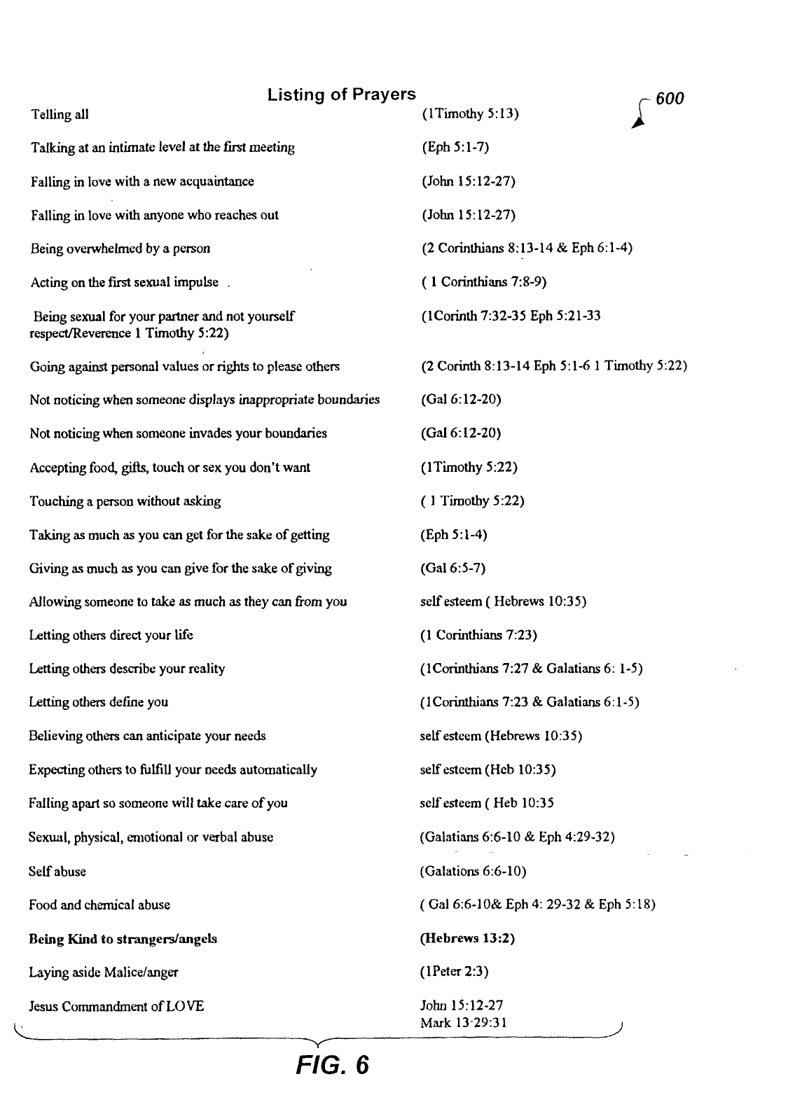

Facilitating the patient's flow of spiritual energy 60 also includes the additional step of utilizing a specific essential oil and incense that correspond to the patient's particular health problem during a therapeutic session. FIG. 5A outlines an essential oils and incense listing that correspond to various physiological and emotional conditions 500. FIG. 5B also lists additional essential oils and incense that are noted in Biblical scripture 510. The therapist would refer to the medical history questionnaire 400 and prepare the appropriate essential oils and incense during the therapeutic session based on the medical history questionnaire 400. Facilitating the patient's flow of spiritual energy also includes the additional step of reciting appropriate scripture corresponding to the patient's particular emotional problems during a therapeutic session. FIG. 6 outlines various scripture verses that correspond to ease various emotional distresses 600. The list includes a description of an emotional problem or situation and a corresponding citation of scripture that can be looked up and read for relief and comfort.

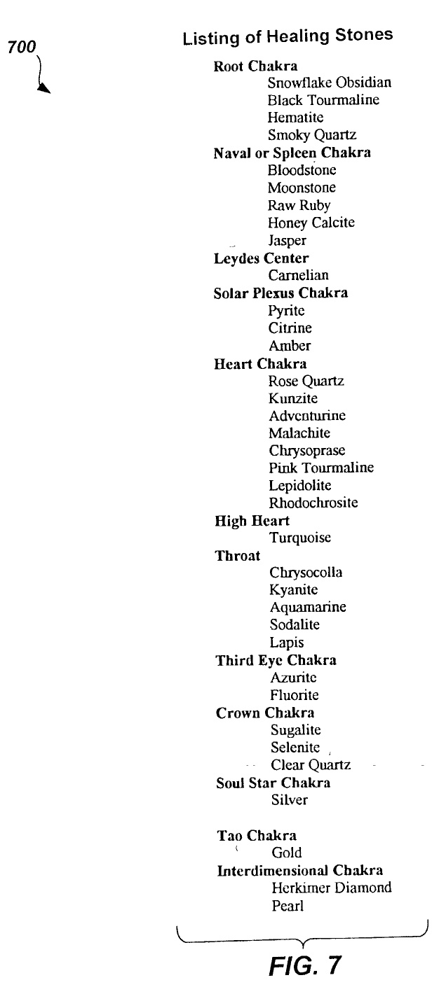

Facilitating the patient's flow of spiritual energy 60 also includes the additional step of utilizing the appropriate healing stone corresponding to the patient's particular chakra and health problem during a therapeutic session. It is believed that certain stones can absorb the negative energy from specific physical and emotional conditions. These healing stones respond and effect particular chakras and other high energy areas of the body and are listed on FIG. 7 700.

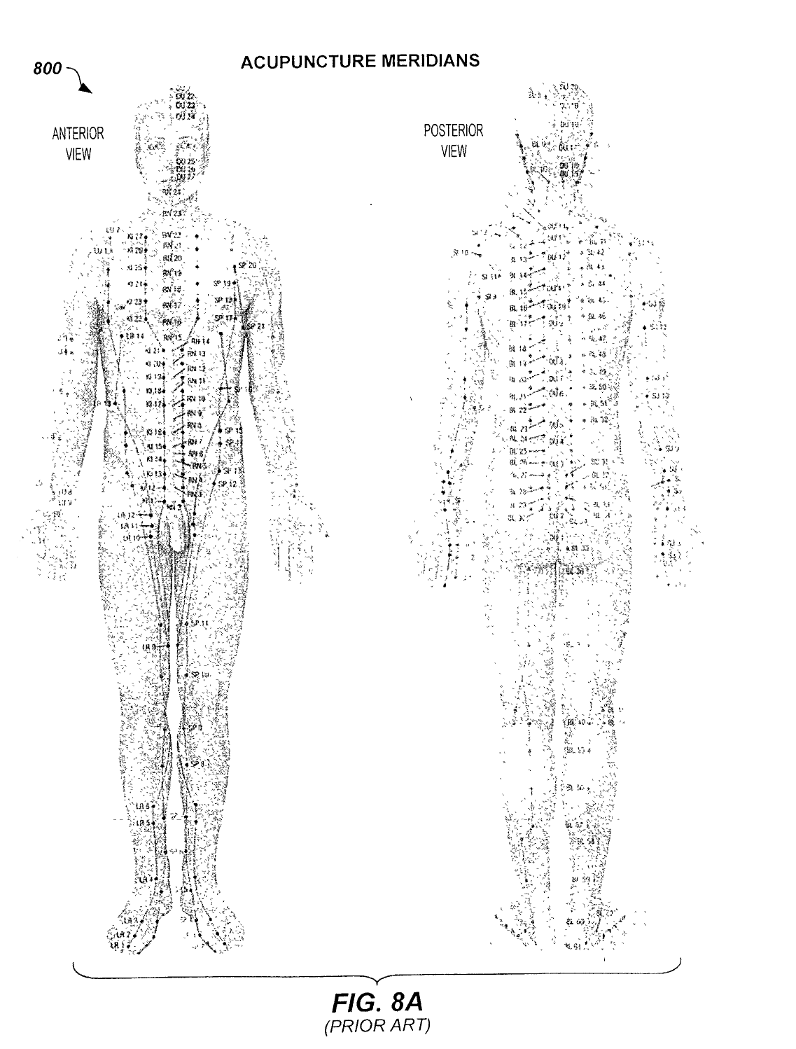

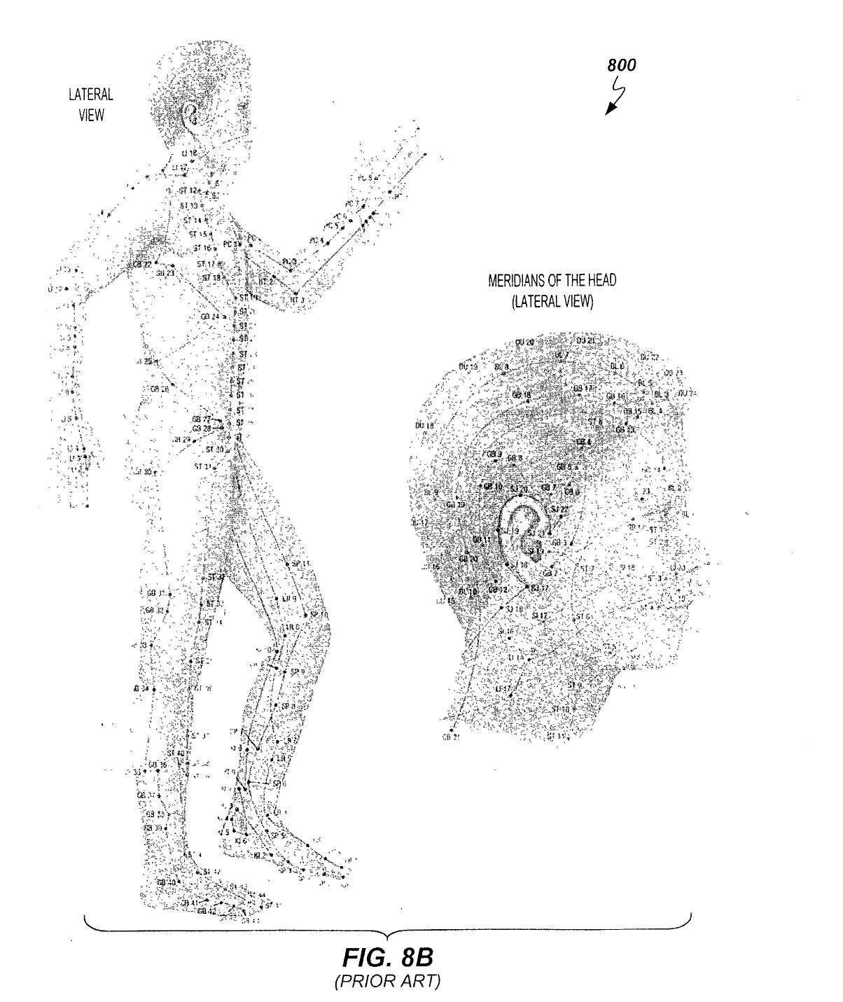

The next step of the overall method is repatterning the patient's areas of spiritual imbalance and impeded energy flow 70 and includes the additional step of utilizing an appropriate massage technique corresponding to the patient's particular health problem during a therapeutic session. The therapist should reference the previously discussed medical history questionnaire 400 and protocol table 200 for addressing any problem areas. This should be done with the additional step of selecting and massaging acupoints along meridians that corresponding to the patient's particular health problems. These acupoints can be located by referencing an acupuncture meridian chart 800, such as the ones in FIG. 8A and FIG. 8B.

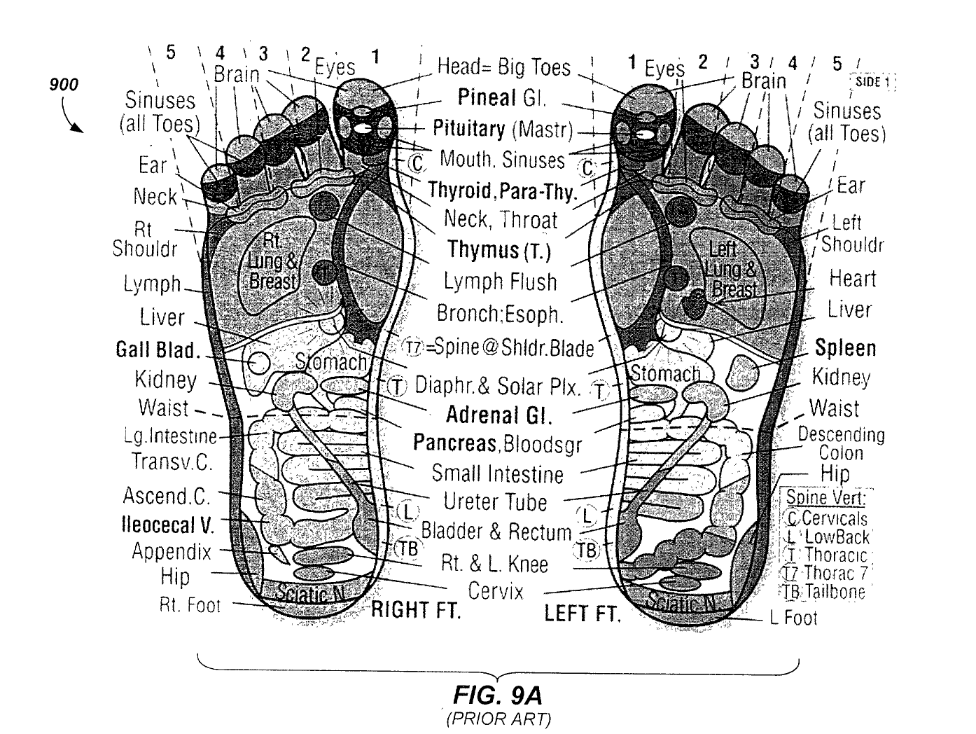

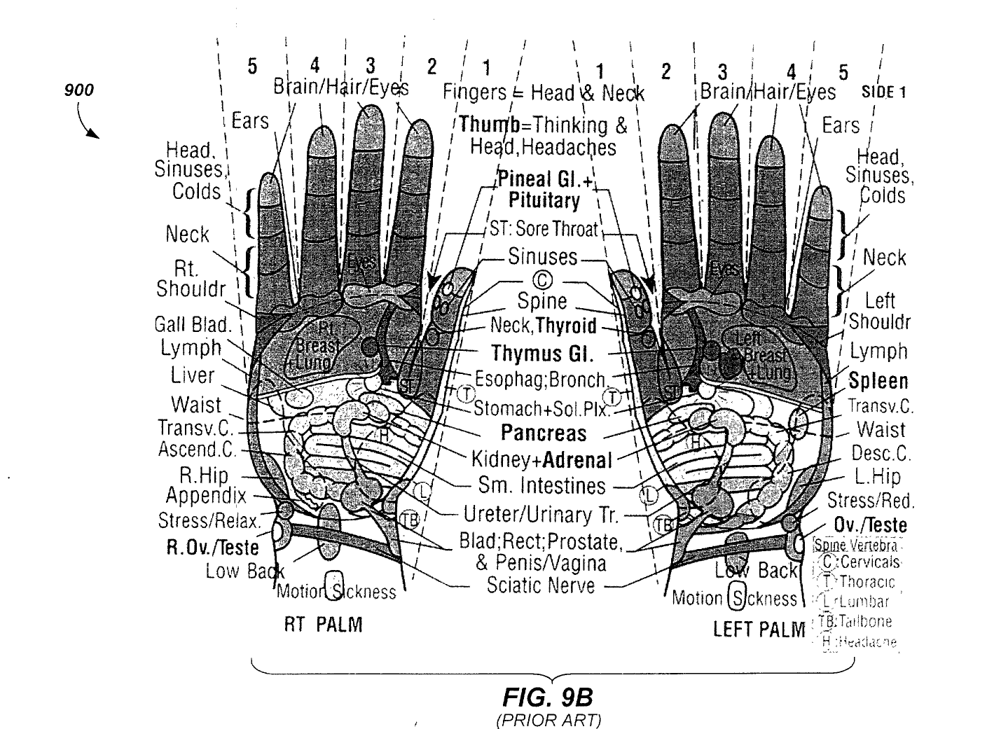

The therapist should have the patient comfortably lie on the therapy table and slowly approach the patient and move into his or her spiritual energy field. The therapist should slowly extend his or her hands and keeping his or her thumbs together and fingers closed, should lightly touch the face plate of the patient. The touch should be similar to a handkerchief slowly touching the face of the patient. The therapist should begin to feel the heat from the patient's face and use the strokes of effleurage, pettrissage, taopotment, vibration, friction, palming, shiatsu and kneading to slowly move down the patient's entire body. Specifically, the therapist should then slowly move to the neck and ears, the shoulders, the left arm, left wrist and left hand, the right arm, right wrist and right hand, the chest, the stomach, the left leg, left ankle and left foot and the right leg, ankle and foot. The therapist will then turn the patient over onto his or her back and slowly move to the back of the head, the back, the buttocks, the right leg, right ankle and right foot and the left leg, left ankle and left foot. The therapist should move down the patient's body in this sequence and should use his or her fingertips to feel for any spiritual energy imbalances at any acupuncture meridians. The repatterning the patient's areas of spiritual imbalance and impeded energy flow 70 should also include non-touch movements such as aura stroking, praying, blowing and singing. Reflexology charts, such as the those in FIGS. 9A and 9B also direct the therapist to massaging corresponding areas of the palms and the bottom of the feet of the patient for particular problem areas.

Once completing these touching and non-touching movements, the therapist should begin disconnection from the client. This can be done by the therapist placing their right hand palm down approximately 6 inches from the patient's head at the crown chakra and closing the chakra by making a rotational motion with their right palm three times counterclockwise. This should be done with the patient's remaining 6 chakras previously discussed. The therapist will then place his or her palms together and silently say “I now separate myself from you and my chakras will now close”. The therapist should then wash his or her hands and disconnect from the energy field of the patient and end the therapeutic session.

The final two steps of the overall method are then completed. These are encouraging the patient's feedback 80 and documenting the results of the therapeutic session and feedback 90. The patient should then be asked how they feel and should voice any opinions that they have about the therapeutic session. The protocol tables 200 can also be used at this time to further educate the patient.

It is to be understood that the present invention is not limited to the embodiment described above, but encompasses any and all embodiments within the scope of the following claims.

I claim:

1. An alternative therapy method for pain management and healing, performed by a therapist on a patient, comprising the steps of:
preparing the therapist, the patient and their environment for a therapeutic session;
assessing the patient's spiritual energy field;
having the therapist explain to the patient what procedures will be done during the therapeutic session;
obtaining verbal permission from the patient to perform the procedures during the therapeutic session;
facilitating the patient's flow of spiritual energy;
repatterning the patient's areas of spiritual imbalance and impeded energy flow;
encouraging the patient's feedback; and
documenting the results of the therapeutic session and the patient's feedback.

2. The method according to claim 1, wherein preparing the therapist includes the step of the therapist performing a visualization technique to clear his or her thoughts and clear any negative spiritual energy in the patient's and therapist's environment during the therapeutic session.

3. The method according to claim 1, wherein preparing the patient's and the therapist's environment involves using incense to clear negative spiritual energy in their environment during the therapeutic session.

4. The method according to claim 1, wherein a clearing solution is used to clear negative spiritual energy in the patient's and therapist's environment during and after the therapeutic session.

5. The method according to claim 4, wherein the clearing solution is alcohol and Lavender essential oil.

6. The method according to claim 1, wherein an open container of sea salt and water is placed in the patient's and therapist's environment to clear negative spiritual energy during the therapeutic session.

7. The method according to claim 1, wherein assessing the patient's spiritual energy flow is done by the therapist using a chakra table.

8. The method according to claim 1, wherein assessing the patient's spiritual energy flow involves the additional step of the therapist obtaining and analyzing the patient's previous medical history.

9. The method according to claim 1, wherein facilitating the patient's flow of spiritual energy including the additional step of utilizing a specific essential oil and incense that correspond to the patient's particular health problem during the therapeutic session.

10. The method according to claim 1, wherein facilitating the patient's flow of spiritual energy includes the additional step of reciting appropriate Bible passages corresponding to the patient's particular emotional problem during the therapeutic session.

11. The method according to claim 1, wherein facilitating the patient's flow of spiritual energy includes the additional step of utilizing the appropriate healing stone corresponding to the patient's particular chakra and health problem during the therapeutic session.

12. The method according to claim 1, wherein repatterning the patient's areas of spiritual imbalance and impeded energy flow includes the additional step of utilizing an appropriate massage technique corresponding to the patient's particular health problem during the therapeutic session.

13. The method according to claim 12, wherein the appropriate massage technique is Swedish, Shiatsu, Reiki and reflexology.

14. The method according to claim 1, wherein repatterning the patient's areas of spiritual imbalance and impeded energy flow includes the additional step of selecting and massaging acupoints along meridians that corresponding to the patient's particular health problem during the therapeutic session.

15. The method according to claim 1, wherein repatterning the patient's areas of spiritual imbalance and impeded energy flow includes the additional step of the therapist washing his or her hands before and after the therapeutic session.

16. The method according to claim 1, wherein repatterning the patient's areas of spiritual imbalance and impeded energy flow includes non-touch movements such as aura stroking, praying, blowing and singing.
